(19) United States
(12) Patent Application Publication
(10) Pub. No.: US 2024/0083919 A1
(43) Pub. Date: Mar. 14, 2024

(54) Metal Complexes and Methods for the Same

(71) Applicant: Colgate-Palmolive Company, New York, NY (US)

(72) Inventors: Stanislav JARACZ, Somerset, NJ (US); Hongwei SHEN, Holmdel, NJ (US)

(73) Assignee: Colgate-Palmolive Company, New York, NY (US)

(21) Appl. No.: 18/259,606

(22) PCT Filed: Dec. 29, 2021
(86) PCT No.: PCT/US2021/065457

§ 371 Date: Jun. 28, 2023

Related U.S. Application Data
(60) Provisional application No. 63/132,424, filed on Dec. 30, 2020.

(51) Int. Cl. C07F 3/06; A61K 8/58; A61Q 5/00

(52) U.S. Cl. CPC C07F 3/06; A61K 8/58; A61Q 5/006

(57) ABSTRACT

Metal complexes, personal care compositions incorporating the metal complexes, and methods for the same are disclosed. The metal complex may include a caprylhydroxamic acid ligand and a metal ion coupled with one another.

### CROSS-REFERENCE TO RELATED APPLICATION

This application claims the benefit of priority from U.S. Provisional Application No. 63/132,424, filed Dec. 30, 2020, the contents of which are hereby incorporated herein by reference in their entirety.

### BACKGROUND

Infections of the nail and skin (e.g., scalp) are known to pose problems in dermatology. The sources of these infections may often be from microbes, such as fungi, viruses, yeast, and/or bacteria. For example, athlete's foot or tinea pedis is a relatively common fungal infection of the skin. Current treatments for treating tinea pedis often include topical treatments, such as topical application of conventional creams, lotions, gels or solutions including antimicrobial agents. While conventional antimicrobial agents have demonstrated efficacy for treating a wide range of conditions caused by various microbes, an increase in awareness regarding antimicrobial resistance has resulted in a corresponding increased interest in discovering new antimicrobial agents.

What is needed, then, are improved antimicrobial agents and methods for treating, inhibiting, or preventing conditions with the antimicrobial agents.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing a metal complex. The metal complex may include a caprylhydroxamic acid ligand and a metal ion coupled with one another.

In at least one implementation, the metal complex may have a ratio of the caprylhydroxamic acid ligand to the metal ion of from about 1:1 to about 4:1, about 1:1 to about 3:1, about 1:1 to about 2:1, about 1:1 to about 1.5:1, about 1:1, or about 1:2.

In at least one implementation, the metal complex may be represented by structure (1), as further disclosed herein, wherein M may be the metal ion.

In at least one implementation, the metal complex may be represented by structure (2), as further disclosed herein, wherein M may be the metal ion.

In at least one implementation, the metal ion may be or include a transition metal ion.

In at least one implementation, the metal ion may be selected from or selected from the group consisting of a zinc ion, a stannous ion, an iron ion, a copper ion, a ferrous ion, a ferric ion, magnesium ion, silver ion, nickel ion, calcium ion, aluminum ion, and zirconium ion.

In at least one implementation, the metal ion is a zinc ion.

In at least one implementation, the metal complex may be represented by structure (3), as further disclosed herein.

In at least one implementation, the metal complex may be represented by structure (4), as further disclosed herein.

The foregoing and/or other aspects and utilities embodied in the present disclosure may be achieved by providing a method for preparing any one or more of the metal complexes disclosed herein. The method may include contacting a caprylhydroxamic acid or a salt thereof and the metal ion with one another in a solvent. The method may also include heating the solvent including the caprylhydroxamic acid or the salt thereof and the metal ion.

In at least one implementation, the caprylhydroxamic acid may be represented by structure (5), as further disclosed herein.

In at least one implementation, the caprylhydroxamic acid may be represented by structure (6), as further disclosed herein, wherein X may be an alkali metal. For example, X may be sodium.

In at least one implementation, the metal ion may be provide by a metal ion source. In at least one example, the metal ion source may be a zinc salt, more preferably the metal ion source may include one or more of zinc acetate, zinc benzoate, zinc borate, zinc bromide, zinc chloride, zinc formate, zinc gluconate, zinc lactate, zinc laurate, zinc malate, zinc nitrate, zinc perborate, zinc sulfate, zinc sulfamate, zinc tartrate, or mixtures thereof.

The foregoing and/or other aspects and utilities embodied in the present disclosure may be achieved by providing a personal care composition including any one or more of the metal complexes disclosed herein.

The foregoing and/or other aspects and utilities embodied in the present disclosure may be achieved by providing a method for preventing or inhibiting the growth of a microorganism. The method may include contacting any one or more of the metal complexes disclosed herein with the microorganism.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### BRIEF DESCRIPTION OF THE DRAWINGS

The present invention will become more fully understood from the detailed description and the accompanying drawings, wherein:

FIG. 1 illustrates a ¹H NMR spectrum of the CHA-Zn complex prepared in Example 1.

FIG. 2 illustrates a mass spectrum of the CHA-Zn complex prepared in Example 2.

FIG. 3 illustrates a ¹H NMR spectrum of the CHA-Zn complex prepared in Example 2.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

The present inventors have surprisingly and unexpectedly discovered that a caprylhydroxamic acid-metal (CHA-metal) complex may be prepared or formed by contacting a metal ion with caprylhydroxamic acid (CHA) or a salt thereof. The CHA-metal complex may have a ratio of CHA to the metal ion of from about 1:1 to about 4:1, about 1:1 to about 3:1, about 1:1 to about 2:1, about 1:1 to about 1.5:1, about 1:1, or about 1:2. The present inventors have further surprisingly and unexpectedly discovered that the CHA-metal complex exhibits antimicrobial efficacy comparable to conventional antimicrobial agents. Specifically, the present inventors have surprisingly and unexpectedly discovered that the CHA-metal complexes disclosed herein exhibit minimum inhibitory concentrations (MIC) comparable to (e.g., parity or substantially equal to) or less than (e.g., better than) conventional antimicrobial agents. As used herein, the term or expression minimum inhibitory concentration or MIC value may refer to a concentration where a substance, a compound, or a complex inhibits at least 80%, at least 90%, at least 95%, at least 98%, or 100% growth relative to an untreated control.

As discussed above, complexes of a metal ion and caprylhydroxamic acid (CHA) are disclosed herein. As used herein, the term or expression “complex” or “metal complex” or “coordination complex” may refer to a compound in which a central metal atom or ion is bonded to other molecules, ligands, or complexing agents by coordinate covalent bonds. In at least one implementation, the complex of the metal ion and CHA (CHA-metal) may be represented by structure (1) and/or structure (2).

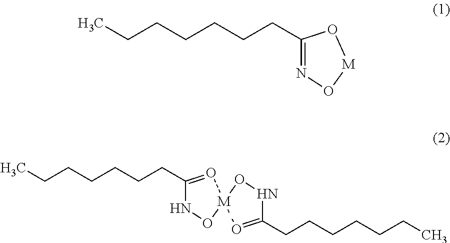

where M may be a metal ion, such as a transition metal ion. Illustrative metal ions may be or include, but are not limited to, a zinc ion, a stannous ion, an iron ion, a copper ion, a ferrous ion, a ferric ion, a magnesium ion, a silver ion, a nickel ion, a calcium ion, an aluminum ion, a zirconium ion, or the like, or combinations thereof.

The ratio of the metal ion to the ligand may be from about 1:1 to about 1:4. For example, the ratio of the metal ion to the CHA ligand may be from about 1:1 to about 1:4, about 1:1 to about 1:3, about 1:1 to about 1:2, about 1.5:1 to about 1:2, or about 1:2. For example, each metal ion may form a complex with one, two, three, four, or more ligands.

In an exemplary implementation, the complexes disclosed herein include complexes formed between CHA and zinc. For example, the complexes disclosed herein include those represented by structure (3), (4), or combinations thereof:

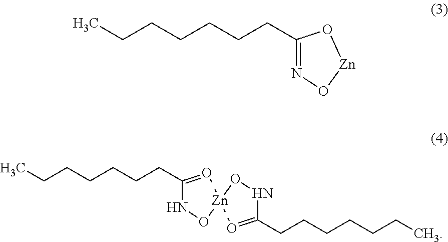

The complexes disclosed herein may be formed or synthesized by combining, mixing, stirring, or otherwise contacting CHA with the metal ion or a source of the metal ion in an appropriate solvent to prepare a reaction mixture. In at least one embodiment, the reaction mixture may be heated at a temperature sufficient to form the complex. In another embodiment, the reaction mixture may not be heated to form the complex. In at least one embodiment, the reaction mixture may be agitated, stirred, or otherwise mixed to prepare the complex.

The CHA-metal may have a solubility in an aqueous solution or solvent of from about 0.01 g to about 5 g per 100 g water. It should be appreciated that the solubility of the CHA-metal may be facility with the addition of one or more organic molecules, such as an alcohol, glycerin, or combinations thereof. The solubility of the CHA-metal complex may also be facilitated with a co-solvent.

The CHA utilized herein may be an amino acid derived from coconut oil. As such, the CHA may be considered a natural ingredient, as it is derived from a natural source. The CHA disclosed herein may be utilized in the form of the amino acid, as represented by structure (5). The CHA disclosed herein may also be utilized in the form of a salt, such as a sodium (Na) salt, as represented by structure (6).

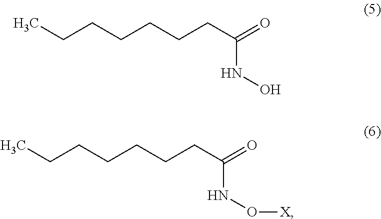

where X may be an alkali metal (e.g., lithium, sodium potassium, etc.).

The metal ion may be any metal ion capable of or configured to attract the electrons of the oxygen atoms of the CHA ligand to form the complex. The metal ion may be provided by a metal ion source, which may generally be a salt. Illustrative salts may be on include, but are not limited to, acetates, benzoates, borates, bromides, chlorides, formats, gluconates, lactates, laurates, malates, nitrates, perborates, sulfates, sulfamates, tartrates, or mixtures thereof. For example, the metal ion may be a zinc ion and the source of the zinc ion may be or include, but are not limited to, zinc acetate, zinc benzoate, zinc borate, zinc bromide, zinc chloride, zinc formate, zinc gluconate, zinc lactate, zinc laurate, zinc malate, zinc nitrate, zinc perborate, zinc sulfate, zinc sulfamate, zinc tartrate, or mixtures thereof. In a preferred implementation, the metal ion is zinc.

The amount of the CHA and the metal ion present in the reaction mixture may vary. In at least one implementation, the ratio of the CHA to the metal ion in the reaction mixture for preparing the complex may be from about 0.5:1 to about 4:1. For example, the ratio of the CHA to the metal ion in the reaction mixture may be from about 0.5:1, about 1:1, about 1.5:1, about 2:1, about 3:1, or about 3.5:1 to about 4:1. In another example, the ratio of the CHA to the metal ion in the reaction mixture may be from about 0.5:1 to about 1:1, about 1.5:1, about 2:1, about 3:1, about 3.5:1, or about 4:1.

The CHA and the metal ion may be combined with one another in the reaction mixture for a time sufficient to form the complex. As discussed above, heat may be applied to the reaction mixture to facilitate the formation of the CHA-metal complex.

The present disclosure may provide compositions incorporating any one or more of the CHA-metal complexes disclosed herein. For example, the present disclosure provides compositions incorporating any one or more of the complexes represented by structures (1)-(4). Compositions incorporating the complexes disclosed herein may be or include a personal care product or a personal care composition. As used herein, the term or expression “personal care composition” may refer to a composition for topical application to skin of mammals, especially humans. The personal care composition may generally be a leave-on personal care composition or rinse off personal care composition, and may include any product applied to a human body. The personal care composition is preferably a leave-on personal care composition. The personal care composition may be in any suitable form. Illustrative forms of the personal care composition may be or include, but is not limited to, a liquid, a lotion, a cream, a foam, a scrub, a gel, a soap bar, a toner, a substance or composition applied with an implement or via a face mask, or the like. Illustrative personal care compositions may be or include, but are not limited to, cleansers, leave-on skin lotions or creams, emulsion, shampoos, conditioners, shower gels, antiperspirants, deodorants, depilatories, lipsticks, foundations, mascara, sunless tanners, sunscreen lotions, body washes, soaps, including bar soaps and liquid soaps (e.g., liquid hand soaps), face washes, moisturizers, serums, spot treatments, cosmetics, or the like.

The present disclosure may provide methods for inhibiting the growth of a microorganism, killing a microorganism, or both. The method may include contacting the microorganism with any one or more of the complexes disclosed herein. For example, the method may include any one of the complexes represented by structures (1)-(4) with the microorganism. As used herein, the term or expression “microorganism” may refer to one or more fungi, yeasts, viruses, bacteria, parasites, or combinations thereof. The microorganism may be inside an animal, on a surface of the animal (e.g., skin), or both. In an exemplary embodiment, the animal may be or include, but is not limited to, any one or more of human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, turkey, any domesticated animal, or combinations thereof. In an exemplary implementation, the animal is a human, and the microorganism is on an external surface of the human. For example, the microorganism is on the skin and/or hair of the human.

In at least one implementation, the microorganism includes a fungus, a yeast, or combinations thereof. The fungus or yeast may be selected from one or more of Candida species, Trichophyton species, Microsporium species, Aspergillus species, Cryptococcus species, Blastomyces species, Cocciodiodes species, Histoplasma species, Paracoccidioides species, Phycomycetes species, Malassezia species, Fusarium species, Epidermophyton species, Scytalidium species, Scopulariopsis species, Alternaria species, Penicillium species, Phialophora species, Rhizopus species, Scedosporium species, Zygomycetes class, or combinations thereof. In another exemplary implementation, the fungus or yeast may be selected from Aspergilus fumigatus (A. fumigatus), Blastomyces dermatitidis, Candida Albicans (C. albicans, both fluconazole sensitive and resistant strains), Candida glabrata (C. glabrata), Candida krusei (C. krusei), Cryptococcus neoformans (C. neoformans), Candida parapsilosis (C. parapsilosis), Candida tropicalis (C. tropicalis), Cocciodiodes immitis, Epidermophyton floccosum (E. floccosum), Fusarium solani (F. solani), Histoplasma capsulatum, Malassezia furfur (M. furfur), Malassezia pachydermatis (M. pachydermatis), Malassezia sympodialis (M. sympodialis), Microsporum audouinii (M. audouinii), Microsporum canis (M. canis), Microsporum gypseum (M. gypseum), Paracoccidioides brasiliensis and Phycomycetes spp, Trichophyton mentagrophytes (T. mentagrophytes), Trichophyton rubrum (T. rubrum), Trichophyton tonsurans (T. tonsurans). In another exemplary embodiment, the fungus or yeast may be selected from Trichophyton concentricum, T. violaceum, T. schoenleinii, T. verrucosum, T. soudanense, Microsporum gypseum, M. equinum, Candida guilliermondii, Malassezia globosa, M. obtuse, M. restricta, M. slooffiae, Aspergillus flavus, or combinations thereof. In another exemplary implementation, the fungus or yeast may be selected from dermatophytes, Trichophyton, Microsporum, Epidermophyton, yeast-like fungi, or the like, or combinations thereof. In a preferred implementation, the microorganism includes Trichophyton rubrum and/or Malassezia furfur.

In an exemplary implementation, the microorganism includes a bacteria. The bacteria may be a gram-positive bacteria. Illustrative gram-positive bacteria species may be or include, but are not limited to, one or more of Staphylococcus species, Streptococcus species, Bacillus species, Mycobacterium species, Corynebacterium species (Propionibacterium species), Clostridium species, Actinomyces species, Enterococcus species, Streptomyces species, or combinations thereof. In at least one implementation, the microorganism may be or include a gram-negative bacteria. Illustrative gram-negative bacteria species may be or include, but are not limited to, Acinetobacter species, Neisseria species, Pseudomonas species, Brucella species, Agrobacterium species, Bordetella species, Escherichia species, Shigella species, Yersinia species, Salmonella species, Klebsiella species, Enterobacter species, Haemophilus species, Pasteurella species, Streptobacillus species, Spirochetal species, Campylobacter species, Vibrio species, Helicobacter species, or combinations thereof. In another exemplary implementation, the bacterium may include one or more of Propionibacterium acnes; Staphylococcus aureus; Staphylococcus epidermidis, Staphylococcus saprophyticus; Streptococcus pyogenes; Streptococcus agalactiae; Streptococcus pneumoniae; Enterococcus faecalis; Enterococcus faecium; Bacillus anthracis; Mycobacterium avium-intracellulare; Mycobacterium tuberculosis, Acinetobacter baumanii; Corynebacterium diphtheria; Clostridium perfringens; Clostridium botulinum; Clostridium tetania; Neisseria gonorrhoeae; Neisseria meningitidis; Pseudomonas aeruginosa; Legionella pneumophila; Escherichia coli; Yersinia pestis; Haemophilus influenzae; Helicobacter pylori; Campylobacter fetus; Campylobacter jejuni; Vibrio cholerae; Vibrio parahemolyticus; Treponema pallidum; Actinomyces israelii; Rickettsia prowazekii; Rickettsia rickettsii; Chlamydia trachomatis; Chlamydia psittaci; Brucella abortus; Agrobacterium tumefaciens; Francisella tularensis, or combinations thereof.

The present disclosure may provide methods for treating or preventing an infection, or both. The method may include administering to the animal a therapeutically effective amount of any one or more of the complexes disclosed herein to treat or prevent the infection. In at least one implementation, the complex includes any one of the complexes represented by structures (1)-(4). Administering the complexes may include topical administration of any one or more of the complexes. As used herein, the term or expression “topical administration” may refer to the application of an agent to an external surface, such as skin, nail, hair, claw, hoof, or the like, or combinations thereof. Topical administration may include application of the complex or agent to skin, nail, hair, claw or hoof, or to a broken, raw or open wound of skin, nail, hair, claw or hoof.

The present disclosure may provide methods for treating or preventing athlete's foot or tinea pedis. For example, the present disclosure may provide methods for treating, preventing, or inhibiting the growth of one or more microbes associated with tinea pedis, such as Trichophyton rubrum. The method may include administering to the animal a therapeutically effective amount of any one or more of the complexes disclosed herein. In at least one implementation, the complex includes any one of the complexes represented by structures (1)-(4). Administering the complexes may include topical administration of any one or more of the complexes. For example, administering the complexes may include topical administration of any one or more of the complexes on skin infected with Trichophyton rubrum.

The present disclosure may provide methods for treating, inhibiting, or preventing dandruff. For example, the present disclosure may provide methods for treating, preventing, or inhibiting the growth of one or more microbes associated with dandruff, such as Malassezia furfur. The method may include administering to the animal a therapeutically effective amount of any one or more of the complexes disclosed herein. In at least one implementation, the complex includes any one of the complexes represented by structures (1)-(4). Administering the complexes may include topical administration of any one or more of the complexes. For example, administering the complexes may include topical administration of any one or more of the complexes on skin (e.g., scalp) infected with Malassezia furfur.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

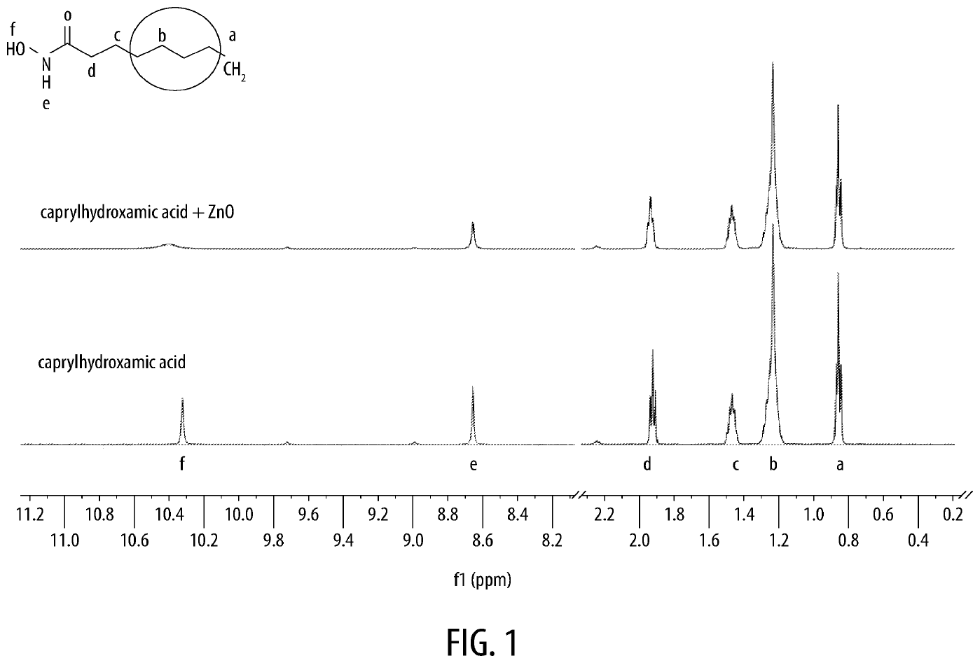

An exemplary caprylhydroxamic-metal complex (CHA-metal complex) was prepared and characterized. Specifically, a caprylhydroxamic-zinc complex was prepared. To prepare the CHA-metal complex, an oil soluble ligand caprylhydroxamic acid (CHA) and zinc ions in the form of zinc oxide (ZnO) were contacted or combined with one another. The ratio of CHA to the ZnO was about 2:1. The CHA and the ZnO were contacted with one another in water and heated at about 50° C. for about 24 hours. After preparation, the CHA-metal complex was characterized via i H NMR; the results of which are illustrated in FIG. 1.

As illustrated in FIG. 1, the CHA-metal complex exhibited peaks or chemical shifts at about 0.86 ppm, about 1.23 ppm, about 1.46 ppm, about 1.94 ppm, about 8.66 ppm, and about 10.40 ppm. As further illustrated in FIG. 1, prior to combining the CHA with the zinc ions, the chemical shift associated with the hydroxyl group (at position 1) was relatively greater than and relatively more narrow after combining the CHA with the zinc ions. It should be appreciated that this indicates the formation of the complex between the zinc ions and the CHA at the hydroxyl functional group.

The molecular weight of the CHA-metal complex was calculated with ¹H NMR diffusion measurements. The theoretical molecular weight of the CHA-Zn complex was about 224.5 g/mol. The calculated molecular weight of the CHA-Zn complex was about 220.6 g/mol, which suggests a stoichiometric ratio of the CHA to the Zn in the complex of about 1:1. Without being bound by theory, it is believed that the synthesis of the CHA-Zn complex proceeded according to chemical equation (7):

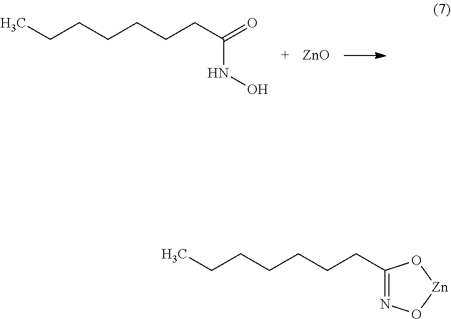

### Example 2

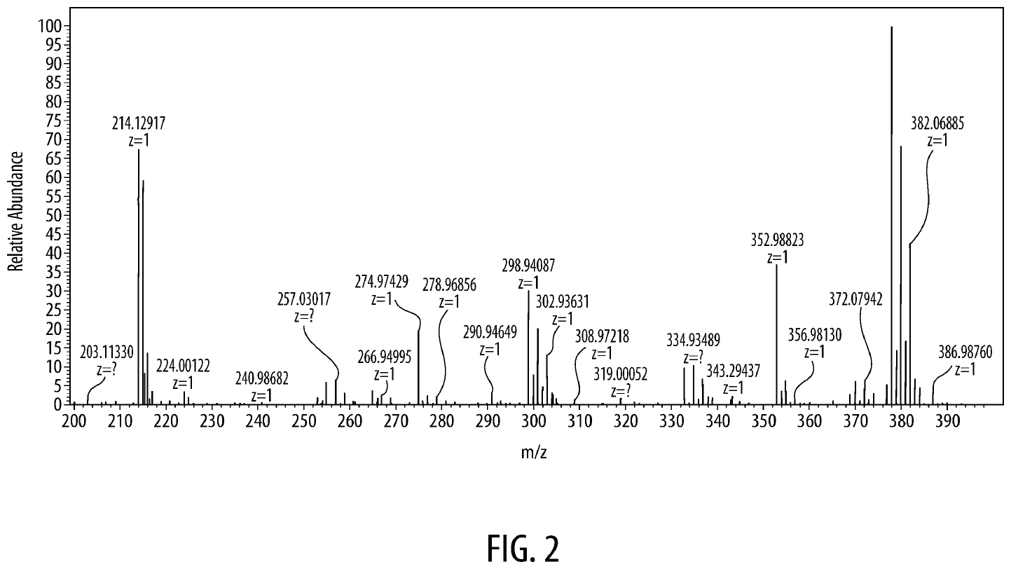

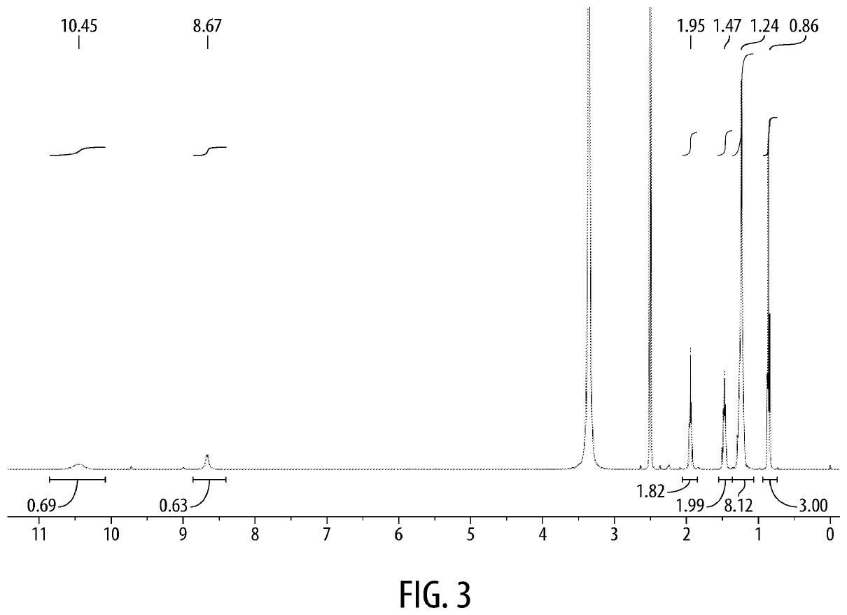

Another exemplary caprylhydroxamic-metal complex (CHA-metal complex) was prepared and characterized. Specifically, a caprylhydroxamic-zinc complex was prepared. To prepare the CHA-metal complex, a soluble salt of caprylhydroxamic acid (CHA), specifically, a sodium salt of CHA and zinc ions in the form of zinc acetate (Zn(CH₃CO₂)₂ or Zn(Ac)₂) were contacted or otherwise combined with one another. The ratio of the sodium salt of CHA to the Zn(Ac)₂ was about 2:1. The CHA and the Zn(Ac)₂ were contacted with one another in water. It was observed that a precipitate immediately formed upon contacting the CHA with the Zn(Ac)₂, which indicated the formation of the complex. After preparation, the CHA-metal complex was characterized via mass spectroscopy and ¹H NMR; the results of which are illustrated in FIGS. 2 and 3, respectively.

As illustrated in FIG. 2, the mass spectroscopy of the complex revealed two peaks with zinc isotopic patterns; namely one peak at about 378 mass-to-charge ratio (m/z) and another peak at about 299 m/z. The mass spectrum of the complex suggested a ratio of the CHA to the Zinc of about 2:1. Without being bound by theory, it is believed that the CHA-Zn complex proceeded or was prepared according to chemical equation (8)

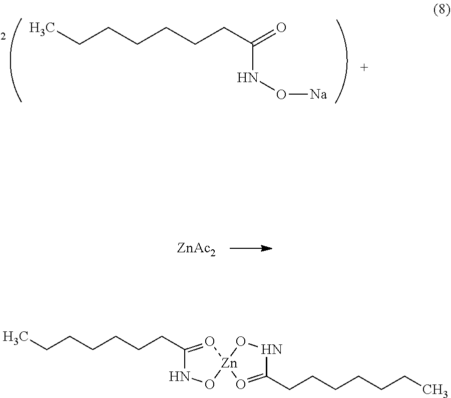

As illustrated in FIG. 3, the CHA-metal complex exhibited peaks or chemical shifts at about 0.86 ppm, about 1.23 ppm, about 1.46 ppm, about 1.94 ppm, about 8.66 ppm, and about 10.40 ppm, similar to the complex prepared in Example 1. The molecular weight of the CHA-metal complex was calculated with ¹H NMR diffusion measurements. The theoretical molecular weights of the CHA-Zn complex at a ratio of 2:1 and 1:1 are about 383.8 g/mol and about 224.5 g/mol, respectively. The calculated molecular weight of the complex was about 246.8 g/mol. This suggested a stoichiometric ratio of the CHA to the Zn in the complex of about 1.55:1.

Without being bound by theory, it is believed that the CHA-Zn complex formed included a mixture of complexes. Particularly, it appears that the mixture included at least two complexes, a first complex having a ratio of the ligand to the metal ion of 2:1 and a second complex having a ratio of the ligand to the metal ion of 1:1.

### Example 3

Another exemplary caprylhydroxamic-metal complex (CHA-metal complex) was prepared and characterized. Specifically, a caprylhydroxamic-zinc complex was prepared. To prepare the CHA-metal complex, an oil soluble ligand caprylhydroxamic acid (CHA) and zinc ions in the form of zinc nitrate (Zn(NO₃)₂) were contacted or combined with one another in an aqueous solvent. The ratio of CHA to the Zn(NO₃)₂ was about 2:1. The CHA and the Zn(NO₃)₂ were contacted with one another in water and heated at about 50° C. for about 24 hours to form a precipitate or the CHA-metal complex. After preparation, the CHA-metal complex was characterized via ¹H NMR and MS. The results of the ¹H NMR and MS suggested a 2:1 ratio of CHA to Zn.

### Example 4

The CHA-Zn complex prepared in Example 3 was challenged with a microbe to determine its efficacy against the microbe. Specifically, the CHA-Zn complex prepared in Example 3 (1) was evaluated to determine the minimum inhibitory concentration (MIC) when challenged with Trichophyton rubrum, a fungus attributed to skin conditions such as athlete's foot (tinea pedis). Comparative samples of CHA alone (2), Zn(NO₃)₂ alone (3), as well as a reference agent zinc pyrithione (ZPT) (4) were also evaluated to determine the respective MICs when challenged with Trichophyton rubrum.

To evaluate the MIC, a series of doubling dilutions was prepared for each of the CHA-Zn complex (1) and the comparative examples (2)-(4). A suspension of Trichophyton rubrum was also prepared and exposed to each of the series of doubling dilutions. The samples were incubated at about 35° C. for 3 to 14 days or until growth was apparent in a growth control tube. Following incubation, each of the samples were visually examined for growth on the basis of turbidity. The MIC of each was recorded at the highest dilution of product that completely inhibited growth of the microorganism as detected by an unaided eye. The results of the MIC analysis is summarized in Table 1. It should be appreciated that a relatively lower MIC value indicates a relatively greater efficacy against the microbe.

TABLE 1
MIC of CHA-Zn Complex (1) and Comparative Examples (2)-(4)
for Trichophyton rubrum

| | | Initial | Final | |
|---|---|---|---|---|
| | | Concentration | Dilution | MIC |
| | Sample | (%) | (Ratio) | (ppm) |
| (1) | CHA-Zn complex | 0.5 | 64 | 78 |
| (2) | CHA | 1 | 64 | 156 |
| (3) | Zinc Nitrate (Zn(NO₃)₂) | 1 | 4 | 2500 |
| (4) | Zinc pyrithione (ZPT) | 1 | 512 | 20 |

As demonstrated in Table 1, the MIC values of CHA alone (2) and zinc nitrate alone (3) were about 156 ppm and 2500 ppm, respectively. The CHA-Zn complex, however, exhibited a MIC value of about 78 ppm, which is significantly greater that the expected MIC value based on the respective MIC values observed in the CHA and zinc nitrate alone. The results are both surprising and unexpected.

### Example 5

The CHA-Zn complex prepared in Example 3 was evaluated to determine the minimum inhibitory concentration (MIC) when challenged with Malassezia furfur, a fungus attributed to skin conditions such as the formation of dandruff. Comparative samples of CHA alone (2), Zn(NO3)2 alone (3), as well as a reference antifungal agent Piroctone Olamine (5) were also evaluated to determine the respective MICs when challenged with Malassezia furfur.

To evaluate the MIC, a series of doubling dilutions was prepared for each of the CHA-Zn complex (1) and the comparative examples (2), (3), and (5). A suspension of Malassezia furfur was also prepared and exposed to each of the series of doubling dilutions. The samples were incubated at about 35° C. for 3 to 5 days or until growth was apparent in a growth control tube. Following incubation, each of the samples were visually examined for growth on the basis of turbidity. The MIC of each was recorded at the highest dilution of product that completely inhibited growth of the microorganism as detected by an unaided eye. The results of the MIC analysis is summarized in Table 2.

TABLE 2
MIC of CHA-Zn Complex (1) and Comparative Examples (2), (3),
and (5) for Malassezia furfur

| | | Initial | Final | |
|---|---|---|---|---|
| | | Concen- | Dilution | MIC |
| | Sample | tration (%) | (Ratio) | (ppm) |
| (1) | CHA-Zn complex | 0.5 | 64 | 78 |
| (2) | CHA | 1 | 32 | 312 |
| (3) | Zinc Nitrate (Zn(NO₃)₂) | 1 | 2 | >5000 |
| (5) | Piroctone Olamine | 1 | 64 | 156 |

As demonstrated in Table 2, the MIC values of CHA alone (2) and zinc nitrate alone (3) were about 312 ppm and >5000 ppm, respectively. The CHA-Zn complex, however, exhibited a MIC value of about 78 ppm, which is significantly greater that the expected MIC value based on the respective MIC values observed in the CHA and zinc nitrate alone. Further, the MIC value of the CHA-Zn complex (1) was significantly less than the MIC value for the reference agent, piroctone olamine, which is a conventional antidandruff agent. The results are both surprising and unexpected.

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

1. A metal complex, comprising a caprylhydroxamic acid ligand and a metal ion.

2. The metal complex of claim 1, wherein the metal complex comprises a ratio of the caprylhydroxamic acid ligand to the metal ion of from about 1:1 to about 4:1, about 1:1 to about 3:1, about 1:1 to about 2:1, about 1:1 to about 1.5:1, about 1:1, or about 1:2.

3. The metal complex of claim 1, wherein the metal complex is represented by structure (1): 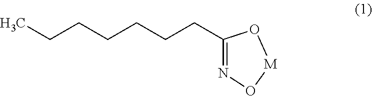
where M is the metal ion.

4. The metal complex of claim 1, wherein the metal complex is represented by structure (2): 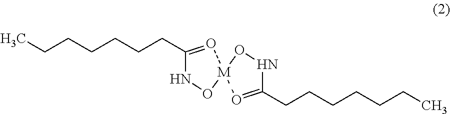
where M is the metal ion.

5. The metal complex of claim 1, wherein the metal ion comprises a transition metal ion.

6. The metal complex of claim 1, wherein the metal ion is selected from a zinc ion, a stannous ion, an iron ion, a copper ion, a ferrous ion, a ferric ion, magnesium ion, silver ion, nickel ion, calcium ion, aluminum ion, or zirconium ion.

7. The metal complex of claim 1, wherein the metal ion is a zinc ion.

8. The metal complex of claim 1, wherein the metal complex is represented by structure (3):

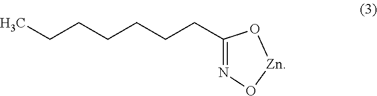

9. The metal complex of claim 1, wherein the metal complex is represented by structure (4):

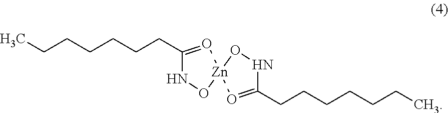

10. A method for preparing the metal complex of claim 1, the method comprising contacting a caprylhydroxamic acid or a salt thereof and the metal ion with one another in a solvent, optionally, heating the solvent.

11. The method of claim 10, wherein the caprylhydroxamic acid is represented by the structure (5):

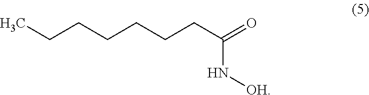

12. The method of claim 10, wherein the caprylhydroxamic acid is represented by the structure (6): 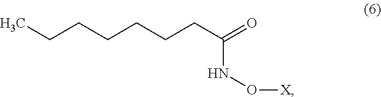
where X is an alkali metal, preferably X is sodium.

13. The method of claim 10, wherein the metal ion is provide provided by a metal ion source, preferably the metal ion source is a zinc salt, more preferably the metal ion source comprises one or more of zinc acetate, zinc benzoate, zinc borate, zinc bromide, zinc chloride, zinc formate, zinc gluconate, zinc lactate, zinc laurate, zinc malate, zinc nitrate, zinc perborate, zinc sulfate, zinc sulfamate, zinc tartrate, or mixtures thereof.

14. A personal care composition comprising the metal complex of claim 1.

15. A method for preventing or inhibiting the growth of a microorganism, the method comprising contacting the metal complex of claim 1 with the microorganism.
